(19) United States
(12) Patent Application Publication
(10) Pub. No.: US 2025/0228831 A1
(43) Pub. Date: Jul. 17, 2025

(54) METHOD TO PREVENT AND TREAT GLAUCOMA BY CALCIUM CHANNEL BLOCKERS, ANGIOTENSIN CONVERTING ENZYME INHIBITORS, AND ANGIOTENSIN RECEPTOR BLOCKERS

(71) Applicant: Assa Weinberg, Los Angeles, CA (US)

(72) Inventor: Assa Weinberg, Los Angeles, CA (US)

(21) Appl. No.: 19/170,382

(22) Filed: Apr. 4, 2025

Related U.S. Application Data
(63) Continuation of application No. PCT/US2023/034581, filed on Oct. 5, 2023.
(60) Provisional application No. 63/378,718, filed on Oct. 7, 2022.

(51) Int. Cl. A61K 31/4422; A61K 31/04; A61K 31/133; A61K 31/138; A61K 31/165; A61K 31/166; A61K 31/167; A61K 31/18; A61K 31/216; A61K 31/34; A61K 31/353; A61K 31/401; A61K 31/403; A61K 31/404; A61K 31/41; A61K 31/4178; A61K 31/4184; A61K 31/4245; A61K 31/472; A61K 31/50; A61K 31/549; A61K 31/55; A61K 31/675; A61P 27/06

(52) U.S. Cl. CPC A61K 31/4422; A61K 31/04; A61K 31/133; A61K 31/138; A61K 31/165; A61K 31/166; A61K 31/167; A61K 31/18; A61K 31/216; A61K 31/34; A61K 31/353; A61K 31/401; A61K 31/403; A61K 31/404; A61K 31/41; A61K 31/4178; A61K 31/4184; A61K 31/4245; A61K 31/472; A61K 31/50; A61K 31/549; A61K 31/55; A61K 31/675; A61P 27/06

(57) ABSTRACT

Methods are provided to prevent and to treat glaucoma by using Calcium channel blockers, Angiotensin-Converting Enzyme (ACE) Inhibitors, or angiotensin receptor blockers (ARB), and more particularly, to a method to prevent and treat glaucoma by using calcium channel blockers, Angiotensin-Converting Enzyme Inhibitors, or angiotensin receptor blockers that may not need to be taken orally, but may also be administered by ophthalmic preparation directly onto or into the eye where glaucoma is formed, to increase or grow the capillary network and augment the blood supply to the anatomy of the eye.

### INCORPORATION BY REFERENCE TO RELATED APPLICATION

This application is a continuation of U.S. National Phase of International No. PCT/US2023/034581, filed Oct. 5, 2023, and claims the benefit of U.S. Provisional Application No. 63/378,718, filed Oct. 7, 2022. The aforementioned application is incorporated by reference herein in its entirety, and each is hereby expressly made a part of this specification.

### FIELD OF THE DISCLOSURE

Methods are provided to prevent, treat, ameliorate, or reduce glaucoma by using calcium channel blockers, Angiotensin-Converting Enzyme (ACE) inhibitors, or angiotensin receptor blockers (ARB), and more particularly, to a method to prevent and treat glaucoma by using calcium channel blockers, ACE inhibitors, or angiotensin receptor blockers that need not be taken orally, but may also be administered by ophthalmic preparation directly onto or into the eye where the conditions for glaucoma occur, to increase or grow the capillary network and augment the blood supply to and within the anatomy of the eye, and the optic nerve.

### BACKGROUND OF THE DISCLOSURE

Glaucoma was first described in 400 BC by Hippocrates, who described it as a blinding disease that occurred in the elderly. The father of medicine was impressed by the patient's pupil's gray-blue color, which translates to Glaycoseis in Greek. Richard Banister, a British ophthalmologist in 1622, described the disease as visual loss from increased tension in the eyeball. The invention of the ophthalmoscope in 1850 made it possible to observe changes in the fundus, which led Donder to discover that high intraocular pressure causes blindness. The invention made further progress in the tonometer. The first effective surgical treatment, an iridectomy, was carried out in 1856 by von Graffe. The first drug treatment started with the discovery of pilocarpine in 1875.

Worldwide, glaucoma is the second-leading cause of blindness after cataracts. Approximately 70 million people have glaucoma globally, with about two million patients in the United States. The overall incidence of glaucoma in the population is 0.5 percent. Nearly 0 percent of the population presents signs or symptoms of glaucoma for individuals aged 40 to 49 years, but increasing up to 5.4 percent of the population by age 80. Glaucoma is a group of eye diseases that result in damage to the optic nerve (or retina) and cause vision loss. Glaucoma can present gradually or suddenly.

While the underlying cause of glaucoma remains unclear, several theories hypothesize on the origins of the pathology. In open-angle glaucoma, generally, the drainage angle for fluid within the eye remains open, with less common types of glaucoma, including closed-angle (narrow angle or acute congestive) glaucoma and normal-tension glaucoma. Open-angle glaucoma develops slowly over time; generally, the patient will not report pain. However, if glaucoma presents suddenly, the condition can involve severe eye pain, blurred vision, mid-dilated pupil, redness of the eye, and nausea. Symptoms of glaucoma may include severe headache, severe eye pain, nausea or vomiting, blurred vision, halos or colored rings around lights, and eye redness. Progression of the disease may damage the optic nerve and may cause vision loss.

While the underlying cause of glaucoma remains unclear, several theories hypothesize on the origins of the pathology. There appears to be some correlation between intraocular pressure and glaucoma. However, the inconsistent relationship between glaucoma symptoms and increased intraocular pressure has generated several competing hypotheses and resulting studies on glaucoma. Risk factors for glaucoma include increasing age, high pressure in the eye, a family history of glaucoma, and use of steroid medication. For eye pressures, a value of 21 mmHg or 2.8 kPa above atmospheric pressure (760 mmHg) is often used as an indicator of glaucoma risk, with higher pressures indicating a greater risk. However, some patients may have high eye pressure for years and never develop damage associated with glaucoma. Optic nerve damage may occur with normal pressure, known as normal-tension glaucoma. Diagnosis of glaucoma is generally achieved by performing a dilated eye examination with an ophthalmoscope to allow doctors to observe any optic nerve damage, as degeneration of axons of the retinal ganglion cells (the optic nerve) is a hallmark of glaucoma.

Screening for glaucoma is usually performed as part of a standard eye examination performed by optometrists and ophthalmologists. Testing for glaucoma includes measurements of the intraocular pressure using tonometry, anterior chamber angle examination, or gonioscopy, as well as examination of the optic nerve to discern visible damage, changes in the cup-to-disc ratio, rim appearance, and vascular change. A formal visual field test is performed. The retinal nerve fiber layer can be assessed with imaging techniques such as optical coherence tomography, scanning laser polarimetry, or scanning laser ophthalmoscopy (Heidelberg retinal tomogram). Visual field loss is the most specific sign of the condition, though this dramatic symptom usually occurs later in the course of the disease.

The most common type of glaucoma is open-angle (including subtypes wide angle and chronic simple) glaucoma. In open-angle glaucoma, the drainage angle for fluid within the eye generally remains open, with less common types of glaucoma, including closed-angle (narrow-angle or acute congestive) glaucoma and normal-tension glaucoma. Open-angle glaucoma develops slowly over time, and generally, the patient will not report pain. However, if glaucoma presents suddenly, the condition can involve severe eye pain, blurred vision, mid-dilated pupil, redness of the eye, and nausea. The mechanism of open-angle glaucoma is hypothesized to be related to the slow exit of aqueous humor through the trabecular meshwork, while in closed-angle glaucoma, the iris blocks the trabecular meshwork. An increase in pressure may compress or otherwise damage the optic nerve.

In open/wide-angle glaucoma, flow is reduced through the trabecular meshwork due to the degeneration and obstruction of the trabecular meshwork, whose original function is to absorb the aqueous humor. Loss of aqueous humor absorption leads to increased resistance and a generally painless buildup of pressure in the eye. In close/narrow-angle glaucoma, the iridocorneal angle may be fully or mostly closed due to, for example, a forward displacement of the iris against the cornea. This forward displacement of the iris would hinder the ability of the aqueous fluid to flow from the posterior to the anterior chamber of the eye. An accumulation of aqueous humor can cause an increase in pressure within the eye and acute pain.

A variety of contributing factors is suspected for glaucoma, including anatomic structure of a patient's eye, eye development, nerve compression trauma, optic nerve blood flow, excitatory neurotransmitter, trophic factor, retinal ganglion cell/axon degeneration, glial support cell, immune system, aging mechanisms of neuron loss, and pressure related severing of the nerve fibers at the scleral edge.

The first drug proscribed to reduce elevated intraocular pressure, pilocarpine, was introduced in the 1870s. Other significant innovations in pharmacological glaucoma therapy were the introduction of beta blocker eye drops in the 1970s and prostaglandin analogs and topical (locally administered) carbonic anhydrase inhibitors in the mid-1990s. Less invasive procedures like small implants have recently supplemented early surgical techniques like iridectomy and fistulating methods. During an ophthalmoscopic exam on a patient presenting with glaucoma, the optic nerve may appear hollowed. The phenomenon of a hollowed optic nerve is described as cupping, and cupping is associated with the loss of axons of the ganglia cells. Most people diagnosed with one of the many types of glaucoma will preserve their vision. However, even some patients who do receive treatment will have degraded vision such that the patients are unable to drive.

While treatments exist, there is no consensus on the primary cause of the disease and eye dysfunction, and there is still a need for a treatment that can prevent, delay, or reverse glaucoma. Glaucoma damage is generally permanent. Usually, pharmacological compositions and/or surgery are only employed to help stop further damage.

### SUMMARY OF THE DISCLOSURE

For one or more of the forgoing reasons, there is a need for a new mode of treatment for glaucoma that increases or grows the capillary network and augments capillary blood supply to the anatomy of the eye.

For one or more of the forgoing reasons, there is a need for a new mode of treatment, amelioration, reduction, or prevention for glaucoma that protects the anatomy of the eye from, and treats, ameliorates, reduces, or prevents the symptoms of, glaucoma, by increasing or growing the capillary network and augmenting the capillary blood supply to the eye.

In particular, there is a need for a method to prevent optic nerve damage and the symptoms of optic nerve damage by increasing the blood supply and the capillary network to the anatomy of the eye where the defects to the anatomy of the eye will form. This may include a method to increase or grow the capillary network and blood supply to the optic nerve and the retina.

For one or more of the forgoing reasons, there is a need for a method that treats the chronic damage to the eye and the symptoms of glaucoma, by increasing or growing the capillary network and augmenting the capillary blood supply to the vulnerable eye anatomy, and to treat the damaged ocular tissue and the symptoms of the damaged ocular tissue. This damaged tissue may include the optic nerve and the retina.

In particular, a method to treat the damaged ocular tissue and the symptoms of damaged ocular tissue by increasing or growing the capillaries network and augmenting the capillary blood supply to the optic nerve region where symptoms associated with glaucoma have occurred. This optic nerve region may include the optic nerve and the retina.

The embodiments are directed to a method for preventing glaucoma by direct administration of a pharmaceutical preparation of calcium channel blockers, ACE inhibitors, or angiotensin receptor blockers to prevent the process and symptoms of glaucoma.

By “glaucoma” used herein, it is meant any process in the eye that causes the appearance and symptoms of glaucoma.

In one method of the disclosure, calcium channel blockers, ACE inhibitors, or angiotensin receptor blockers may be administered directly to the intravitreal space before symptoms form, so as to prevent glaucoma, for example, in men or women with normal lens before the degenerative process starts.

In another embodiment, calcium channel blockers, ACE inhibitors, or angiotensin receptor blockers may be administered directly to the intravitreal space to treat and heal the symptoms of glaucoma.

Calcium channel blockers, ACE inhibitors, or angiotensin receptor blockers may be administered even after symptoms of glaucoma have dissipated.

Calcium channel blockers, ACE inhibitors, and angiotensin receptor blockers are a class of pharmaceutical drugs that when taken orally dilate the arteriolar system.

Contact calcium channel blockers, ACE inhibitors, or angiotensin receptor blockers are a new class of pharmaceutical products. When applied directly to target tissue it increases or grows the capillary network, augments the capillary blood supply, and enhances tissue repair in diverse body membrane tissues. Currently, there are over 20 pharmaceutical patented ACE inhibitor drugs that use this property to treat hypertension, and congestive heart failure. The clinical indication of calcium channel blockers, ACE inhibitors, and angiotensin receptor blockers is therefore limited to the field of cardiovascular diseases. Calcium channel blockers, ACE inhibitors, and angiotensin receptor blockers were extensively studied but their ability to prevent and treat glaucoma remained unknown.

The methods of the embodiments are directed to the use of an application of calcium channel blockers, ACE inhibitors, or angiotensin receptor blockers to the eye lens space. The direct contact of ACE inhibitors with the ocular tissue, and in particular, the intravitreal space, increases grows the capillary network and augments the blood supply to the ocular tissue. It also increases or grows the capillary network and augments the blood supply to the ocular lens territory. The direct contact of ACE inhibitors with the ocular tissue may increase or grow the capillary network and blood supply to the optic nerve and, alternatively or in combination, the retina.

Accordingly, in a generally applicable aspect (i.e., independently combinable with any of the aspects or embodiments identified herein), the calcium channel blocker, ACE inhibitor, or angiotensin receptor blocker may be administered directly to the intravitreal space before symptoms form to prevent glaucoma before the degenerative process begins.

In a generally applicable embodiment (i.e., independently combinable with any of the aspects or embodiments identified herein) of the first aspect, the calcium channel blocker, ACE inhibitor, or angiotensin receptor blocker may be administered directly to the intravitreal space to treat and heal the symptoms of glaucoma.

In a generally applicable embodiment (i.e., independently combinable with any of the aspects or embodiments identified herein) of the first aspect, the calcium channel blocker, ACE inhibitor, or angiotensin receptor blocker may be directly to intravitreal space even after the glaucoma has dissipated and the eye has healed to prevent the recurrence.

Calcium channel blockers, ACE inhibitors, and angiotensin receptor blockers are classes of pharmaceutical drugs when taken orally they dilate the arteriolar system by blocking the activity of the calcium channel receptors, ACE receptors or angiotensin receptors.

Contact calcium channel blockers, ACE inhibitors, and angiotensin receptor blockers are a new class of pharmaceutical products. When applied directly to target tissue they increase or grow the capillary network, augment the capillary blood supply, and enhance tissue repair in diverse body membrane tissues. When in contact with the ocular tissue, these pharmaceutical products may increase or grow the capillary network and blood supply to the optic nerve and, alternatively or in combination, the retina.

Currently, there are over 20 pharmaceutical patented ACE inhibitor or angiotensin receptor blocker drugs that use this property to treat hypertension, and congestive heart failure. The clinical indication of ACE inhibitors or angiotensin receptor blockers is therefore generally limited to the field of cardiovascular diseases.

Calcium channel blockers, ACE inhibitors and angiotensin receptor blockers were extensively studied but their ability to prevent and treat glaucoma remained unknown.

The use of application of calcium channel blockers, ACE inhibitors or angiotensin receptor blockers to the lens (vitreous) area of the eye or the intravitreal space is provided. The direct contact with the tissue of the eye increases or grows the capillary network and augments the blood supply to the lens.

Additionally, the use of application of calcium channel blockers, ACE inhibitors or angiotensin receptor blockers to the optic nerve and, alternatively or in combination, the retina is provided. The direct contact with the tissue of the eye increases or grows the capillary network and augments the blood supply to the optic nerve and or the retina.

The new class may be used for the prevention and treatment of glaucoma or other syndromes associated with glaucoma.

### DETAILED DESCRIPTION OF THE DISCLOSURE

The following discussion addresses a number of embodiments and applications of the present disclosure. The beneficial features of the present disclosure will be evident from the described embodiments. It is to be understood that the present disclosure is not limited to such specific applications and that numerous implementations of the present disclosure may be realized. All references to patents, patent applications, and non-patent publications mentioned in the specification are hereby incorporated by reference, in their entireties.

In some aspects, a method is provided herein to treat, prevent, reduce, ameliorate, or reduce glaucoma in a subject. In some embodiments, treating, preventing, reducing, ameliorating, or reducing glaucoma includes treating, preventing, reducing, ameliorating, or reducing one or more signs or symptoms of glaucoma. In some embodiments, a composition or a pharmaceutical composition is provided to the subject to treat, prevent, reduce, ameliorate or reduce glaucoma in the subject. In some embodiments, the composition or pharmaceutical composition includes a contact neo-vasodilator. Contact neo-vasodilators are a new class of medication. This disclosure describes the use of contact neo-vasodilators such as valsartan, a known angiotensin receptor blocker or enalapril, a known ACE inhibitor, or calcium channel blockers used in the treatment of hypertension and congestive heart failure, for the prevention and treatment of glaucoma.

In some embodiments, ACE inhibitors such as ramiprilat, enalapril, benazepril, captopril, enalapril, moexipril, perindopril, quinapril, trandolapril lisinopril, ramipril, or fosinopril or angiotensin receptor blockers such as valsartan, telmisartan, olmesartan, losartan, irbesartan, candesartan and azilsartan, when administered directly to the eye, may be effective for treating, preventing, reducing, ameliorating, or preventing glaucoma.

In some embodiments, Ramiprilat, or other non-sulfhydryl ACE inhibitors with antihypertensive activity may have been used in treatment of high blood pressure, heart failure, or diabetic kidney disease, when applied in a pharmacological composition in an effective amount, directly into the eye, by direct administration or intravitreal injection, may be effective for treating, preventing, reducing, ameliorating, or preventing glaucoma. In some embodiments, the injection may be to the posterior chamber of the eye. Without wanting to be bound by theory it is believed that an ACE inhibitor, such as ramipril or ramiprilat grows capillary networks in the eye and increases or grows the capillary blood supply, reduces the intraocular pressure, repairs the intraocular pressure, and repairs the optic nerve in multiple form glaucoma. Ramiprilat chemical structure is shown below:

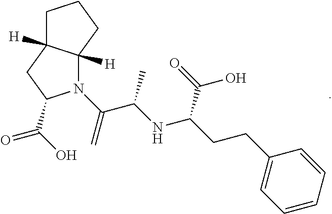 [SMILES: C=C([C@H](C)N[C@@H](CCc1ccccc1)C(=O)O)N1[C@H](C(=O)O)C[C@@H]2CCC[C@@H]21]

In some embodiments, Valsartan, or other angiotensin receptor blockers, or Enalapril, or other ACE inhibitors, which previously may have been used in treatment of high blood pressure and or congestive heart failure, when applied in a pharmacological composition in an effective amount, directly into the eye, by direct administration or intravitreal injection, may be effective for treating, preventing, reducing, ameliorating, or preventing glaucoma. In some embodiments, the injection may be to the posterior chamber of the eye.

In some embodiments, Angiotensin receptor blockers such as Valsartan, telmisartan, olmesartan, losartan, irbesartan, candesartan, eprosartan, sacubitril/valsartan, and azilsartan, when administered directly to the eye, may be effective for treating, preventing, reducing, ameliorating, or preventing glaucoma.

In some embodiments, a pharmacological composition as used herein, may include a pharmaceutical preparation according to the disclosure. In some embodiments, the pharmaceutical composition may include, but is not limited to, one or more ACE inhibitors, calcium channel blockers, or angiotensin receptor blockers and a suitable non-toxic pharmaceutical carrier.

Effective amount as used herein is an amount of the pharmaceutical composition of calcium channel blockers, ACE inhibitors, or angiotensin receptor blockers that is effective for treating the glaucoma. An amount of calcium channel blockers, ACE inhibitors, or angiotensin receptor blockers that is suitable for direct administration or intravitreal injection to the eye. In some embodiments, the injection may be to the posterior chamber of the eye.

A method is provided of applying a pharmaceutical preparation in an effective amount of one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), directly to the tissue of the eye or into the eye via intravitreal injection to treat or prevent glaucoma. In some embodiments, the injection may be to the posterior chamber of the eye.

The pharmacological preparation can comprise a calcium channel blocker. The calcium channel blocker can be in a suitable nontoxic pharmacological carrier. The pharmacological preparation can comprise an ACE inhibitor. The ACE inhibitor can be in a suitable nontoxic pharmacological carrier. The pharmacological preparation can comprise an angiotensin receptor blocker. The angiotensin receptor blocker can be in a suitable nontoxic pharmacological carrier.

In some embodiments, an effective amount for treatment or prevention of glaucoma is administered. In some embodiments, an amount of calcium channel blocker that is suitable for treatment by intravitreal injection or direct application to the tissue of the eye is administered. In some embodiments, the injection may be to the posterior chamber of the eye.

In some embodiments, an effective amount for treatment, reduction, amelioration, or prevention of glaucoma is administered an ACE inhibitor. An amount of ACE inhibitor that is suitable for treatment by intravitreal injection or direct application to the tissue of the eye is administered. In some embodiments, the injection may be to the posterior chamber of the eye.

In some embodiments, an effective amount for treatment, reduction, amelioration, or prevention of glaucoma is administered one or more angiotensin receptor blockers. An amount of angiotensin receptor blocker that is suitable for treatment by intravitreal injection or direct application to the tissue of the eye is administered. In some embodiments, the injection may be to the posterior chamber of the eye.

In some embodiments, an effective amount for treatment, reduction, amelioration, or prevention of glaucoma is administered one or more contact vasodilators. Contact vasodilators (e.g., calcium channel blockers, ACE inhibitors, angiotensin receptor blockers, nitrates, alpha blockers, beta blockers, hydralazine, and/or angiotensin receptor-neprilysin inhibitors) are a new class of pharmaceutical medications that increase blood supply, which produces biological changes.

In some embodiments, the treatment, reduction, or prevention of glaucoma, these changes can include one or more of increasing the blood supply to the vitreous tissue, the optic nerve, the choroid, the sclera, the retina, and the ocular lens. These biological changes may include a change in fluid movement in the anterior chamber and may include a change in fluid movement in the vitreous chamber.

In some embodiments, the treatment, reduction, or prevention of glaucoma, includes administration of one or more calcium channel blockers. Calcium channel blockers are a class of pharmaceutical drugs that disrupt the entry of calcium molecules through the L type voltage operated channels to cardiac muscle and blood vessels cells. The blockage of calcium entry causes the relief of arterial spasm. Currently there are 70 pharmaceutical patented calcium channel blocker drugs that use this property to treat hypertension, angina pectoris and cardiac arrhythmia. The clinical indication for the therapeutic use of calcium channel blockers was therefore limited, until now, to the field of cardiovascular diseases only. Calcium channel blockers were extensively studied but their ability to prevent and or to treat glaucoma remained heretofore unknown.

Accordingly, new uses are provided of contact-applied calcium channel blockers for application to the tissue of the eye (e.g., vitreous tissue) or for intravitreal or intraocular injection. In some embodiments, the injection may be to the posterior chamber of the eye. The new use may be used for the prevention or treatment of glaucoma. No trial of topical calcium channel blockers for the prevention or treatment of glaucoma has heretofore been published. Contact calcium channel blockers are a part of contact-vasodilators, a new class of medication. The use is provided of contact neo-vasodilators such as nifedipine, a known calcium channel blocker used in the treatment of hypertension, for the prevention and treatment glaucoma. Nifedipine, amlodipine, felodipine, isradipine, nicardipine, nisoldipine and clevidipine are in a class of dihydropyridines calcium channel blockers. Verapamil and diltiazem are non-dihydropyridines calcium channel blockers. When applied by contact these are very effective drugs for the treatment or prevention of glaucoma.

In some embodiments, the treatment, reduction, or prevention of glaucoma, includes administration of one or more inhibitors of angiotensin converting enzyme (ACE). ACE inhibitors can be employed as vasodilators. Angiotensin II is a chemical produced by the body that primarily circulates in the blood. It causes the muscles surrounding blood vessels to contract, thereby narrowing the vessels. Angiotensin II is formed from angiotensin I in the blood by the enzyme angiotensin converting enzyme (ACE). Angiotensin I in the blood is itself formed from angiotensinogen, a protein produced by the liver and released into the blood. Angiotensin converting enzyme inhibitors (ACE inhibitors) are medications that slow (inhibit) the activity of the enzyme ACE, which decreases the production of angiotensin II. As a result, blood vessels enlarge or dilate. ACE inhibitors include, but are not limited to benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), ramiprilat, and trandolapril (Mavik). Angiotensin II receptor blockers (ARBs) help relax the blood vessels. Angiotensin II receptor blockers block the action of angiotensin II, allowing blood vessels to dilate. Angiotensin receptor blockers include, but are not limited to: azilsartan (Edarbi), candesartan (Atacand), eprosartan, irbesartan (Avapro), losartan (Cozaar), olmesartan (Benicar), telmisartan (Micardis), and valsartan (Diovan).

Other vasodilators are known in the art. These include, but are not limited to nitrates (nitroglycerin, isosorbide mononitrate and isosorbide dinitrate), Alpha blockers (doxazosin (Cardura), prazosin (Minipress), terazosin), Beta blockers (Acebutolol (Sectral), Atenolol (Tenormin), Bisoprolol fumarate (Zebeta), Carvedilol (Coreg)—Combined alpha/beta blocker, Esmilol (Brevibloc), Labetalol (Trandate, Normodyne)—Combined alpha/beta blocker, Metoprolol tartrate (Lopressor) and metoprolol succinate (Toprol-XL), Nadolol (Corgard), Nebivolol (Bystolic), Penbutolol sulfate (Levatol), Propranolol (Inderal), Sotalol (Betapace), HCTZ and bisoprolol (Ziac) is a beta blocker plus diuretic), Hydralazine, and angiotensin receptor-neprilysin inhibitors (ARNi) (Entresto, sacubitril/valsartan).

### Conditions Amenable to Treatment or Prevention

Compositions and methods are provided for the prevention or treatment of glaucoma.

Application of vasodilators (e.g., calcium channel blockers, ACE inhibitors, angiotensin receptor blockers, nitrates, alpha blockers, beta blockers, hydralazine, and/or angiotensin receptor-neprilysin inhibitors), such as Nifedipine or other calcium channel blockers, which previously may have been used in the treatment of high blood pressure, in a pharmacological composition, in an effective amount, in a contact form, such as, but not limited to an oil, liquid preparation or suspension, to the tissue of the eye (vitreous tissue) or for administration by intravitreal injection, can be employed to treat or prevent the symptoms of glaucoma. In some embodiments, the injection may be to the posterior chamber of the eye.

Pharmacological compositions of the embodiments include but are not limited to one or more vasodilators (e.g., calcium channel blockers, ACE inhibitors, angiotensin receptor blockers, nitrates, alpha blockers, beta blockers, hydralazine, and/or angiotensin receptor-neprilysin inhibitors) and a suitable nontoxic pharmaceutical carrier. The pharmaceutical composition in administered in an amount effective for treating glaucoma, e.g., an amount suitable for treatment by direct application to tissue of the eye (vitreous tissue) or by intravitreal injection. In some embodiments, the injection may be to the posterior chamber of the eye.

Glaucoma, associated symptoms, and treatment thereof, or use of ACE inhibitors, Angiotensin receptor blockers, or calcium channel blockers are described in the following references, each of which is incorporated by reference herein in its entirety and each of which is hereby made a part of this specification: Thylefors B, Négrel AD, Pararajasegaram R, Dadzie KY.Global; Thylefors B. The World Health Organization's programme forthe; Congdon N, O'Colmain B, Klaver CC, et al. Causes and prevalence of visual impairment among adults in the United States. Arch; Congdon N, O'Colmain B, Klaver CC, et al. Causes and prevalence of visual impairment among adults in the United States. Arch Ophthalmol 2004; 122:477;

Compositions including one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), optionally in combination with conventional therapies, and associated methods for treatment of glaucoma and related symptoms are provided.

Some embodiments relate to a pharmaceutical composition and method of treatment using the pharmaceutical composition, wherein the pharmaceutical composition comprises at least one calcium channel blocker, for example, a calcium channel blocker selected from the group consisting of amlodipine (Norvasc), diltiazem (Cardizem LA, Tiazac), felodipine (Plendil), isradipine (Dynacirc), nifedipine (Adalat, Procardia), nicardipine (Cardene), nimodipine (Nimotop), nisoldipine (Sular), verapamil (Covera-HS, Verelan PM, Calan), verapamil, diltiazem and nicardipine (Cardene IV). Some embodiments relate to a pharmaceutical composition and method of treatment using the pharmaceutical composition, wherein the pharmaceutical composition comprises at least one ACE inhibitors, for example at least one ACE inhibitor selected from the group consisting of benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), ramiprilat, and trandolapril (Mavik). Some embodiments relate to a pharmaceutical composition and method of treatment using the pharmaceutical composition, wherein the pharmaceutical composition comprises at least one angiotensin receptor blocker, for example at least one angiotensin receptor blocker selected from the group consisting of azilsartan (Edarbi), candesartan (Atacand), eprosartan, irbesartan (Avapro), losartan (Cozaar), olmesartan (Benicar), telmisartan (Micardis), and valsartan (Diovan). In certain embodiments, the pharmaceutical composition is in a form suitable for contact administration, e.g., to tissue of the eye (vitreous tissue) or by intravitreal administration, however other routes of administration are also considered that involve contact of the vasodilator to the tissue to be treated.

The pharmaceutical compositions for treatment of glaucoma can further comprise other pharmaceutically active ingredients. These can include drugs to control pain, for example, nonsteroidal anti-inflammatory drugs such as ibuprofen or naproxen sodium, topical anesthetics such as lidocaine, drugs to fight infections (e.g., antibiotic, antiviral, or antifungal agents). The treatment can be administered in conjunction with other therapies, e.g., the conventional therapies for glaucoma as described elsewhere herein.

The use of topical vasodilators (e.g., calcium channel blockers, ACE inhibitors, angiotensin receptor blockers, nitrates, alpha blockers, beta blockers, hydralazine, and/or angiotensin receptor-neprilysin inhibitors) for treatment of glaucoma is a new class of drugs. The new class may be used for glaucoma, or to enhance efficacy of conventional glaucoma drugs. In one method, the vasodilator may be applied directly to the tissue of the eye, e.g., in a form of an eyedrop. In another embodiment, the vasodilator may be injected directly into the intravitreal space to treat glaucoma. In some embodiments, the injection may be to the posterior chamber of the eye. The vasodilator may be applied even after the glaucoma has been ameliorated to prevent recurrence of glaucoma.

In one method, the vasodilator may be used to treat a specific type of glaucoma. For example, a patient population may be selected from patients displaying open angle glaucoma or closed angle glaucoma. Within open angle glaucoma, the specific type of glaucoma that is selected for treatment may be: primary open angle glaucoma, low pressure glaucoma, congenital glaucoma, and secondary glaucoma. Low pressure glaucoma, which is also called normal pressure glaucoma, is characterized by optic nerve damage and vision loss despite normal intra ocular pressure. In some embodiments, low pressure glaucoma includes optic disc cupping. In some embodiments, low pressure glaucoma includes visual-field loss with the exception of a consistently normal intraocular pressure, for example, less than 22 mm Hg. In some embodiments, low pressure glaucoma is associated with conditions such as migraines. See Raynaud syndrome and sleep apnea, a Primary Open Angle Glaucoma preferred practice guideline Prum BE jr. Ophthalmology 2016 123(11) P41. Without being bound to a single theory of operation, this type of glaucoma could result from a poor blood supply to the optic nerve. One aspect of the disclosure is directed to treating a type of glaucoma by increasing or growing the capillary network and blood supply to the optic nerve. Alternatively, or in combination, the treatment of glaucoma may increase or grow the capillary network and blood supply to the retina. In some embodiments, low pressure glaucoma is treated or reduced by administration of a vasodilator by topical administration. In some embodiments, the topical administration is by ophthalmic drops. In some embodiments, low pressure glaucoma is treated or reduced by administration of a vasodilator by intraocular administration of a vasodilator. In some embodiments, low pressure glaucoma is treated or reduced by administration of a vasodilator by intravitreal administration of a vasodilator. In some embodiments, low pressure glaucoma is treated or reduced by administration of a vasodilator by intrajunctival administration of a vasodilator. In some embodiments, low pressure glaucoma is treated or reduced by administration of a vasodilator by subcutaneous administration of a vasodilator. In some embodiments, low pressure glaucoma is treated or reduced by administration of a vasodilator by sub-Tenon administration of a vasodilator. In some embodiments, low pressure glaucoma is treated or reduced by administration of a vasodilator by intracameral administration of a vasodilator. In some embodiments, low pressure glaucoma is treated or reduced by administration of a vasodilator by intracameral administration of a vasodilator. In some embodiments, low pressure glaucoma is treated or reduced by administration of a vasodilator by retrobulbar administration of a vasodilator.

In some embodiments, a method is provided for to treat, prevent, or reduce primary open angle glaucoma. In some embodiments, primary open angle glaucoma is treated or reduced by administration of a vasodilator by topical administration. In some embodiments, the topical administration is by ophthalmic drops. In some embodiments, primary open angle glaucoma is treated or reduced by administration of a vasodilator by intraocular administration of a vasodilator. In some embodiments, primary open angle glaucoma is treated or reduced by administration of a vasodilator by intravitreal administration of a vasodilator. In some embodiments, primary open angle glaucoma is treated or reduced by administration of a vasodilator by intrajunctival administration of a vasodilator. In some embodiments, primary open angle glaucoma is treated or reduced by administration of a vasodilator by subcutaneous administration of a vasodilator. In some embodiments, primary open angle glaucoma is treated or reduced by administration of a vasodilator by sub-Tenon administration of a vasodilator. In some embodiments, primary open angle glaucoma is treated or reduced by administration of a vasodilator by intracameral administration of a vasodilator. In some embodiments, primary open angle glaucoma is treated or reduced by administration of a vasodilator by intracameral administration of a vasodilator. In some embodiments, primary open angle glaucoma is treated or reduced by administration of a vasodilator by retrobulbar administration of a vasodilator.

In some embodiments, a method is provided for to treat, prevent, or reduce primary secondary glaucoma. In some embodiments, secondary glaucoma is treated or reduced by administration of a vasodilator by topical administration. In some embodiments, the topical administration is by ophthalmic drops. In some embodiments, secondary glaucoma is treated or reduced by administration of a vasodilator by intraocular administration of a vasodilator. In some embodiments, secondary glaucoma is treated or reduced by administration of a vasodilator by intravitreal administration of a vasodilator. In some embodiments, secondary glaucoma is treated or reduced by administration of a vasodilator by intrajunctival administration of a vasodilator. In some embodiments, secondary glaucoma is treated or reduced by administration of a vasodilator by subcutaneous administration of a vasodilator. In some embodiments, secondary glaucoma is treated or reduced by administration of a vasodilator by sub-Tenon administration of a vasodilator. In some embodiments, secondary glaucoma is treated or reduced by administration of a vasodilator by intracameral administration of a vasodilator. In some embodiments, secondary glaucoma is treated or reduced by administration of a vasodilator by intracameral administration of a vasodilator. In some embodiments, secondary glaucoma is treated or reduced by administration of a vasodilator by retrobulbar administration of a vasodilator.

In some embodiments, a method is provided for to treat, prevent, or reduce congenital glaucoma. Congenital glaucoma may occur in infants and very young children due to a congenital development of the drainage angle of the eye. Symptoms of Congenital glaucoma may include watery eyes, photosensitivity, and a tendency to keep the eye closed. Congenital glaucoma may be diagnosed during an exam where a patient's eyes appear larger than normal and/or the cornea appears cloudy. See Kipp MA Childhood Glaucoma Pediatr Clin. North Am,2003 50:89. In some embodiments, congenital glaucoma is treated or reduced by administration of a vasodilator by topical administration. In some embodiments, the topical administration is by ophthalmic drops. In some embodiments, congenital glaucoma is treated or reduced by administration of a vasodilator by intraocular administration of a vasodilator. In some embodiments, congenital glaucoma is treated or reduced by administration of a vasodilator by intravitreal administration of a vasodilator. In some embodiments, congenital glaucoma is treated or reduced by administration of a vasodilator by intrajunctival administration of a vasodilator. In some embodiments, congenital glaucoma is treated or reduced by administration of a vasodilator by subcutaneous administration of a vasodilator. In some embodiments, congenital glaucoma is treated or reduced by administration of a vasodilator by sub-Tenon administration of a vasodilator. In some embodiments, congenital glaucoma is treated or reduced by administration of a vasodilator by intracameral administration of a vasodilator. In some embodiments, congenital glaucoma is treated or reduced by administration of a vasodilator by intracameral administration of a vasodilator. In some embodiments, congenital glaucoma is treated or reduced by administration of a vasodilator by retrobulbar administration of a vasodilator.

In one method, the treatment of glaucoma may treat patients that are asymptomatic with respect to vision loss. For example, a majority of open angle Glaucoma patients worldwide may asymptomatic, without vision loss, and their intra ocular pressure may below 21 mm. However, such patients may already have visible optic nerve damage by ophthalmoscopy. Currently there is no generally recognized treatment for these millions of patients aside from recommending a follow up visit, where again there is no recommended treatment for early stages of the disease. In some embodiments, asymptomatic glaucoma is treated or reduced by administration of a vasodilator by topical administration. In some embodiments, the topical administration is by ophthalmic drops. In some embodiments, asymptomatic glaucoma is treated or reduced by administration of a vasodilator by intraocular administration of a vasodilator. In some embodiments, asymptomatic glaucoma is treated or reduced by administration of a vasodilator by intravitreal administration of a vasodilator. In some embodiments, asymptomatic glaucoma is treated or reduced by administration of a vasodilator by intrajunctival administration of a vasodilator. In some embodiments, asymptomatic glaucoma is treated or reduced by administration of a vasodilator by subcutaneous administration of a vasodilator. In some embodiments, asymptomatic glaucoma is treated or reduced by administration of a vasodilator by sub-Tenon administration of a vasodilator. In some embodiments, asymptomatic glaucoma is treated or reduced by administration of a vasodilator by intracameral administration of a vasodilator. In some embodiments, asymptomatic glaucoma is treated or reduced by administration of a vasodilator by intracameral administration of a vasodilator. In some embodiments, asymptomatic glaucoma is treated or reduced by administration of a vasodilator by retrobulbar administration of a vasodilator.

One aspect of this disclosure is directed to treating those millions of patients that are observed for the first time with optic nerve damage, where, for example, cupping, is first detected or identified as a possibility. As current treatments of glaucoma focus on treating generally irreversible symptoms like blindness, one advantage of some methods of the disclosure may include early-stage treatment of disease. In some embodiments, vision loss may be prevented by treating the progression of the disease. In some embodiments, vision loss may be prevented by limiting the progression of the disease before the disease manifests more severe symptoms. In some embodiments, optic nerve damage is treated or reduced by administration of a vasodilator by topical administration. In some embodiments, the topical administration is by ophthalmic drops. In some embodiments, optic nerve damage is treated or reduced by administration of a vasodilator by intraocular administration of a vasodilator. In some embodiments, optic nerve damage is treated or reduced by administration of a vasodilator by intravitreal administration of a vasodilator. In some embodiments, optic nerve damage is treated or reduced by administration of a vasodilator by intrajunctival administration of a vasodilator. In some embodiments, optic nerve damage is treated or reduced by administration of a vasodilator by subcutaneous administration of a vasodilator. In some embodiments, optic nerve damage is treated or reduced by administration of a vasodilator by sub-Tenon administration of a vasodilator. In some embodiments, optic nerve damage is treated or reduced by administration of a vasodilator by intracameral administration of a vasodilator. In some embodiments, optic nerve damage is treated or reduced by administration of a vasodilator by intracameral administration of a vasodilator. In some embodiments, optic nerve damage is treated or reduced by administration of a vasodilator by retrobulbar administration of a vasodilator.

Aspects of the disclosure relate to a method of growing a capillary network and increasing or growing the capillary blood supply in the eye of a subject. In some embodiments, the method includes administering a pharmaceutical composition comprising ramipril or ramiprilat and a pharmaceutically acceptable excipient to the eye of the subject, thereby treating multiple forms of glaucoma. In some embodiments, the pharmaceutical composition comprises ramiprilat. In some embodiments, administering the pharmaceutical composition repairs the optic nerve of the subject. In some embodiments, the pharmaceutical composition repairs the retina of the subject. In some embodiments, the multiple forms of glaucoma is a primary glaucoma. In some embodiments, is open-angle glaucoma. In some embodiments, the primary glaucoma is normal-tension glaucoma. In some embodiments, the primary glaucoma is angle-closure glaucoma. In some embodiments, the primary glaucoma is congenital glaucoma. In some embodiments, the multiple forms of glaucoma is a secondary glaucoma. In some embodiments, the secondary glaucoma is neovascular glaucoma, pigmentary glaucoma, exfoliation glaucoma, or uveitic glaucoma.

Aspects of the disclosure relate to a method of treating, preventing, reducing, ameliorating diabetic retinopathy in a subject. In some embodiments, the method includes administering a pharmaceutical composition comprising ramipril or ramiprilat and a pharmaceutically acceptable excipient to the subject, thereby treating, preventing, reducing, ameliorating diabetic retinopathy in the subject. In some embodiments, the pharmaceutical composition comprises ramiprilat. In some embodiments, the pharmaceutical composition repairs the retina in subject.

Aspects of the disclosure relate to a method of treating, preventing, reducing, ameliorating age related macular degeneration in a subject. In some embodiments, the method includes administering a pharmaceutical composition comprising ramipril or ramiprilat and a pharmaceutically acceptable excipient to the subject, thereby treating, preventing, reducing, ameliorating diabetic retinopathy in the subject. In some embodiments, the pharmaceutical composition comprises ramiprilat. In some embodiments, the pharmaceutical composition repairs the retina in subject. In some embodiments, the age related macular degeneration is wet (exudative) macular degeneration. In some embodiments, the age related macular degeneration is dry (or atrophic) macular degeneration.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more pharmacologically active ingredients (e.g. vasodilators) disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

The term “anatomy of the eye” refers to all the parts and subparts of and related to the eye. The anatomy of the eye may refer to, but is not limited to, each of the following individually, collectively, or in some combination of Choroid, Cornea, Fovea, Iris, Macula, Lens, Optic Nerve, Pupil, Retina, Sclera, and Vitreous Humor. As, for example, most of the optic nerve structure is outside of any specific ocular globe territory, the term “anatomy of the eye” may refer to parts and subparts of and related to the eye that are not within the eye itself.

It is to be understood that where compounds disclosed herein (e.g., calcium channel blockers, ACE inhibitors, angiotensin receptor blockers, nitrates, alpha blockers, beta blockers, hydralazine, and/or angiotensin receptor-neprilysin inhibitors) have unfilled valencies, then the valencies are to be filled with hydrogens or isotopes thereof, e.g., hydrogen-1 (protium) and hydrogen-2 (deuterium).

It is understood that the compounds described herein (e.g., calcium channel blockers, ACE inhibitors, angiotensin receptor blockers, nitrates, alpha blockers, beta blockers, hydralazine, and/or angiotensin receptor-neprilysin inhibitors) can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein may include crystalline forms (also known as polymorphs, which include the different crystal packing arrangements of the same elemental composition of a compound), amorphous phases, salts, solvates, and hydrates, e.g., of vasodilators. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein (e.g., vasodilators) may exist in unsolvated as well as solvated forms. In general, the solvated forms are considered equivalent to the unsolvated forms for the purposes of the compounds and methods provided herein.

Where a range of values is provided, it is understood that the upper and lower limit, and any intervening value between the upper and lower limit of the range is included.

Any percentages, ratios or other quantities referred to herein are on a weight basis, unless otherwise indicated.

### Pharmaceutical Compositions

The vasodilators (e.g., calcium channel blockers, ACE inhibitors, angiotensin receptor blockers, nitrates, alpha blockers, beta blockers, hydralazine, and/or angiotensin receptor-neprilysin inhibitors) can be prepared by any suitable method known to those in the art. For representative methods, see, for example, Francis A. Carey et al., Advanced Organic Chemistry: Part B: Reaction and Synthesis (5^{th} Ed. 2005).

Formulations including a vasodilator (e.g., a calcium channel blocker, ACE inhibitor and/or angiotensin receptor blocker) and at least one excipient are provided. It is generally preferred to administer the compounds of the embodiments in topical formulations; however, other routes of administration are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the vasodilator (e.g., a calcium channel blocker, ACE inhibitor and/or angiotensin receptor blocker) used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intravitreal, intranasal, and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions described herein are suitable for use in treatment or prevention of glaucoma or associated symptoms. The compositions are suitable for use in any patient where treatment or prevention of glaucoma is desirable.

The vasodilator (e.g., a calcium channel blocker, ACE inhibitor and/or angiotensin receptor blocker) can be employed in various types of formulations. Topical formulations including one or more vasodilators in combination with at least one excipient are provided. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical formulations for application to skin or ocular membranes. The formulation can be provided as an aqueous formulation, or in an anhydrous formulation which may prevent water-based irritant contact dermatitis or stinging sensation upon application. In another embodiment, the composition is formulated such that preservatives need not be employed (e.g., a preservative-free formulation) so as to avoid eye or skin irritation associated with certain preservatives.

To facilitate application, the composition may be provided as an ointment, an oil, a lotion, a paste, a powder, a gel, a solid insert, or a cream. The composition may also include additional ingredients such as a protective agent, an emollient, a humectant, an antibiotic agent, an antifungal agent, an antiviral agent, an antiprotozoal agent, an anesthetic agent, a steroidal anti-inflammatory agent, a non-steroidal anti-inflammatory agent, an antipruritic agent, an antioxidant agent, an anti-histamine agent, a vitamin or vitamin complex, a hormone, an anti-skin atrophy agent, and combinations thereof. In a further embodiment, the composition may avoid animal or cellular-based materials to avoid irritation. The composition can be applied directly to the vitreous tissue of the eye.

Methods of using vasodilator formulations are provided. The compositions may be applied topically, but may also be applied via intravitreal injection. In some embodiments, the injection may be to the posterior chamber of the eye.

Some embodiments include administering vasodilator (e.g., a calcium channel blocker, ACE inhibitor and/or angiotensin receptor blocker) compositions provided herein in topical formulations; however, other routes of administration are also contemplated (e.g., intraocular or the like). Contemplated routes of administration include but are not limited to topical and intraocular. Suitable liquid forms include suspensions, emulsions, solutions, and the like. Unit dosage forms can also be provided, e.g., individual packets with a premeasured amount of the formulation, configured for administration to the tissue on a predetermined schedule (e.g., daily, weekly, etc.). Unit dosage forms configured for administration twice a day can be employed; however, in certain embodiments it can be desirable to configure the unit dosage form for administration once a day, four times a day, or more, or once every other day, every three days, weekly, or less, or on an as-needed basis.

In some embodiments, the topical and intravitreal formulations includes from about 0.001 wt. % or less to about 50 wt. % or more of active ingredient, such as the vasodilator (e.g., a calcium channel blocker, ACE inhibitor and/or angiotensin receptor blocker). In some embodiments, the topical and intravitreal formulation includes from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Compositions and formulations for topical administration to the vitreous tissue of the eye can include gels, drops, sprays, liquids, and aerosols. Conventional pharmaceutical carriers, aqueous or oily bases, thickeners and the like may be employed. Such formulations are typically provided in an eyedropper. A liquid or gel can also be placed using an applicator, e.g., a wand, a sponge, a syringe, or other suitable method.

A topical formulation can be provided in a form of a carrier containing the vasodilator, e.g., 50 ppm or less to 1000, 5000, 10000, 50000, 100000, 500000 ppm or more of the vasodilator. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % or 20 wt. % of the vasodilator. The amount of vasodilator in the base can be adjusted up or down.

Liquids and gels containing the vasodilator, optionally with other components as described herein, can be prepared using techniques as are known in the art for preparing topical formulation. See, e.g., Handbook of Cosmetic Science and Technology, Fourth Edition, edited by André O. Barel, Marc Paye, Howard I. Maibach, CRC Press, 2014, the contents of which is hereby incorporated by reference in its entirety. Various formulations are possible.

For liquid formulations (e.g., gel or lotion forms), a silicone, e.g., a cyclosiloxane or linear silicone (e.g., silicone elastomer), can be employed as a carrier. One type of suitable carrier is a dimethicone crosspolymer gel, e.g., dimethicone crosspolymer in cyclopentasiloxane. Other suitable dimethicone crosspolymers include cyclopentasiloxane, dimethicone/vinyldimethicone crosspolymer; dimethicone, dimethicone/vinyl dimethicone crosspolymer; and isodecane dimethicone/vinyl dimethicone crosspolymer.

In some embodiments, the carrier is present in an amount of from about 80 wt. % to about 95 wt. %, or 82 wt. % to 92 wt. %, e.g., in a topical formulation for application to skin.

Penetration enhancers can be employed to enhance penetration of the vasodilator into tissue. Typical amounts when employed in topical formulations are from 1% by weight to 4% by weight. Typical amounts for anti-irritation agents when employed in topical formulations are from 1% by weight to 4% by weight. Typical amounts for anti-inflammatory agents when employed in topical formulations are from 1% by weight to 4% by weight. Typical amounts for anti-inflammatory agents when employed in topical formulations are from 0.1% by weight to 2% by weight.

In some embodiments, the vasodilator can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration to the tissue of the eye comprise the vasodilator as described herein and a vehicle acceptable for contact with ocular tissue (e.g., vitreous tissue). The vehicle may be aqueous or nonaqueous. The vehicle used in the topical formulation may be in the form of a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the vasodilator into the ocular tissue (e.g., vitreous tissue).

The pharmaceutical excipients used in the topical preparations of the vasodilator compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the vasodilator compositions as described herein may be dissolved or dispersed in a hydrophobic oil phase, and the oil phase may then be emulsified in an aqueous phase comprising water, alone or in combination with lower alcohols, glycerin, and/or glycols. In certain embodiments water is present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes containing the vasodilators as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the vasodilators as described herein.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The vasodilator compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the vasodilator formulations of the embodiments are administered by intraocular injection, it is preferably in the form of a pyrogen-free, parenterally acceptable aqueous solution or oleaginous suspension, emulsion or solution. In some embodiments, the injection may be to the posterior chamber of the eye. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The vasodilator formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

### Kits for Administration of Compositions

Some embodiments of the methods and compositions provided herein include kits comprising vasodilators provided herein. In some embodiments, kits can be provided to an administering physician, other health care professional, a patient, or a caregiver. In some embodiments, a kit comprises a container which contains the vasodilator(s) in a suitable topical formulation, and instructions for administering the composition to a subject. The kit can optionally also contain one or more additional therapeutic or other agents. For example, a kit containing a vasodilator blocker in topical form can be provided along with other agents such as topical antibiotics or topical anesthetics. The kit may contain the vasodilator in bulk form, or can contain separate doses of the vasodilator for serial or sequential administration. The kit can optionally contain one or more diagnostic tools, administration tools, and/or instructions for use, e.g., syringes for intravitreal injection. In some embodiments, the injection may be to the posterior chamber of the eye. The kit can contain suitable delivery devices, such as, syringes, pump dispensers, wands, single dose packets, and the like, along with instructions for administering the vasodilator compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

The topical formulation for administration to tissue of the eye, in addition to the vasodilator, can contain other ingredients.

While topical administration of the vasodilator disclosed herein can advantageously be employed, in certain embodiments other routes of administration are also contemplated, such as intravitreal or intraocular injection. In some embodiments, the injection may be to the posterior chamber of the eye.

The vasodilator compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The vasodilator compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal, intravitreal, and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the vasodilator may be combined as the active ingredient in intimate admixture with a pharmaceutical carrier according to conventional pharmaceutical compounding techniques. The excipients are preferably minimized so as to ensure administration of an appropriate amount of vasodilator in a compact format. The carrier can take a wide variety of forms depending on the form of preparation desired for administration. Thus, the vasodilator compositions provided herein can be presented as discrete units suitable for administration each containing a predetermined amount of the active ingredient. Further, the vasodilator compositions can be presented as an oil, as a solution, as a suspension in an aqueous liquid, as a non-aqueous liquid, as an oil-in-water emulsion, or as a water-in-oil liquid emulsion, similar to the topical formulations described elsewhere herein, but using components suitable for human contact or consumption. In addition to the common dosage forms set out above, the vasodilator compositions provided herein can also be administered by controlled release and/or delivery devices. The vasodilator compositions can be prepared by any of the methods of pharmacy. In general, such methods include a step of bringing into association the active ingredient with the carrier that constitutes one or more necessary ingredients. In general, the vasodilator compositions are prepared by uniformly and intimately admixing the vasodilator ingredient(s) with liquid carriers or finely divided solid carriers or both. The product can then be conveniently shaped into the desired presentation.

A vasodilator formulation may also be administered in a local manner, for example, via injection of the vasodilator composition directly into a target area, e.g., in a depot or sustained release formulation intravitrealy or intraocularly. Furthermore, a targeted drug delivery system for the vasodilator may be used, for example, in a liposome coated with a tissue specific antibody.

The vasodilator compositions may contain the vasodilator in an amount effective for the desired therapeutic effect. In some embodiments, the vasodilator compositions are in a unit dosage form and comprise from about 0.1 mg or less to about 5000 mg or more of vasodilator per unit dosage form. In further embodiments, the vasodilator compositions comprise from about 1 to about 500 mg per unit dosage form or from about 500 to 5000 mg per unit dosage form of vasodilator. Such amounts can be selected depending upon the vasodilator employed. Such dosage forms may be solid, semisolid, liquid, an emulsion, or adapted for delivery via aerosol or the like.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Vasodilator compositions provided herein can be prepared as solutions or suspensions of the vasodilator in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Vasodilator compositions provided herein suitable for injectable use include sterile aqueous solutions or dispersions. Furthermore, the vasodilator compositions can be in the form of sterile powders for the extemporaneous preparation of such sterile injectable solutions or dispersions. The vasodilator compositions may be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the vasodilator formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood or other bodily fluids of the intended recipient. Vasodilator compositions can also be prepared in powder or liquid concentrate form for dilution.

Contemplated herein are vasodilator compositions including one or more vasodilators as described herein in combination with at least one additional active agent, e.g., an antibiotic. The vasodilator and the at least one additional active agent(s) may be present in a single formulation or in multiple formulations provided together, or may be unformulated. In some embodiments, the vasodilator can be administered with one or more additional agents together in a single composition. For example, the vasodilator can be administered in one composition, and at least one of the additional agents can be administered in a second composition. In a further embodiment, the vasodilator and the at least one additional active agent(s) are co-packaged in a kit. For example, a drug manufacturer, a drug reseller, a physician, a compounding shop, or a pharmacist can provide a kit comprising the vasodilator in combination with another product or component for delivery to a patient. Such additional components can include anti-infective agents, anti-inflammatory agents, anesthetics, or the like.

Some embodiments described herein relate to compositions of vasodilator, which can include a therapeutically effective amount of the vasodilator described herein and a pharmaceutically acceptable carrier, diluent, excipient or combination thereof. The vasodilator composition can include the vasodilator in an amount for example, >1%, ≥2%, ≥3%, ≥4%, ≥5%, ≥6%, ≥7%, ≥8%, ≥9%, ≥10%, ≥20%, ≥30%, >40%, ≥50%, ≥60%, ≥70%, ≥80%, ≥90%, ≥95%, or ≥98% of the composition.

### EXAMPLES

### Example 1

A patient is diagnosed with glaucoma. A composition comprising the calcium channel blocker nifedipine is directly applied to the vitreous surface of one eye while the other eye is left untreated. The treated eye is observed to have reduced symptoms of glaucoma than the untreated eye.

### Example 2

A patient is diagnosed with glaucoma. A composition comprising the ACE inhibitor enalapril is directly applied to the vitreous surface of one eye while the other eye is left untreated. The treated eye is observed to have reduced symptoms of glaucoma than the untreated eye.

### Example 3

A patient is diagnosed with glaucoma. A composition comprising the angiotensin receptor blocker losartan is directly applied to the vitreous surface of one eye while the other eye is left untreated. The treated eye is observed to have reduced symptoms of glaucoma than the untreated eye.

### Example 4

A patient is successfully treated for glaucoma in both eyes. A composition comprising the calcium channel blocker nifedipine is directly applied to the vitreous surface of one eye while the other eye is left untreated. The treated eye is observed to not develop glaucoma, while the untreated eye exhibits signs of recurrence of glaucoma.

### Example 5

A patient is successfully treated for glaucoma in both eyes. A composition comprising the ACE inhibitor enalapril is directly applied to the vitreous surface of one eye while the other eye is left untreated. The treated eye is observed to not develop glaucoma, while the untreated eye exhibits signs of recurrence of glaucoma.

### Example 6

A patient is successfully treated for glaucoma. A composition comprising the angiotensin receptor blocker losartan is directly applied to the vitreous surface of one eye while the other eye is left untreated. The treated eye is observed to not develop glaucoma, while the untreated eye exhibits signs of recurrence of glaucoma.

### Example 7

A patient is diagnosed with glaucoma. A composition comprising the calcium channel blocker nifedipine is applied to the eye by intravitreal or intraocular injection while the other eye is left untreated. The treated eye is observed to have reduced symptoms of glaucoma than the untreated eye.

### Example 8

A patient is diagnosed with glaucoma. A composition comprising the ACE inhibitor enalapril is applied to the eye by intravitreal or intraocular injection while the other eye is left untreated. The treated eye is observed to have reduced symptoms of glaucoma than the untreated eye.

### Example 9

A patient is diagnosed with glaucoma. A composition comprising the angiotensin receptor blocker losartan is applied to the eye by intravitreal or intraocular injection while the other eye is left untreated. The treated eye is observed to have reduced symptoms of glaucoma than the untreated eye.

### Example 10

A patient is successfully treated for glaucoma in both eyes. A composition comprising the calcium channel blocker nifedipine is applied to the eye by intravitreal or intraocular injection while the other eye is left untreated. The treated eye is observed to not develop glaucoma, while the untreated eye exhibits signs of recurrence of glaucoma.

### Example 11

A patient is successfully treated for glaucoma in both eyes. A composition comprising the ACE inhibitor enalapril is applied to the eye by intravitreal or intraocular injection while the other eye is left untreated. The treated eye is observed to not develop glaucoma, while the untreated eye exhibits signs of recurrence of glaucoma.

### Example 12

A patient is successfully treated for glaucoma. A composition comprising the angiotensin receptor blocker losartan is applied to the eye by intravitreal or intraocular injection while the other eye is left untreated. The treated eye is observed to not develop glaucoma, while the untreated eye exhibits signs of recurrence of glaucoma.

### General Statement of Utility

Within the confines of experimental use and with patient consent, vasodilators as described in the current application have been applied to patient's eyes for the treatment, inter alia, of glaucoma. The patients were monitored during follow-up appointments and the progression of glaucoma was monitored and improvements to the patients' conditions was observed. Improvements to the patients' conditions included a reduction in symptoms of glaucoma.

### Specific Statement of Utility

In particular, the group of compounds consisting of nifedipine, isradipine, felodipine, amlodipine, nicardipine, and clevidipine are of particular interest for the treatment of glaucoma. Based on prior experience and observation during the treatment of patients with these compounds, compounds of this group are expected to be highly effective when applied to the eye.

Without being bound to a single theory of operation, the application of compounds described herein is believed to increase blood flow to the eye. The additional blood flow provides many benefits to the anatomy of the eye, and is expected to restore or help maintain the function of the eye. The function of the eye can be evaluated in a variety of ways. In one aspect, Glaucoma may be treated by reducing the interocular pressure of the eye. In one aspect, Glaucoma may be treated by increasing the amount of flow of vitreous fluid between the anterior and posterior chambers of the eye. In another aspect, a method of treating glaucoma may include a method to increase or grow the capillary network and blood supply to the optic nerve and the retina. Damage to the optic nerve may not necessarily require high interocular pressure, and a method of treating glaucoma may include a method to increase or grow the capillary network and blood supply to the optic nerve and the retina such that interocular pressure does not rise above normal pressure levels.

### Exemplary Pharmaceutical Compositions and Methods

Pharmaceutical Composition 1: A pharmaceutical composition for the treatment or prophylaxis of glaucoma, comprising: at least one vasodilator; and at least one pharmaceutical excipient.

Pharmaceutical Composition 2: Pharmaceutical Composition 1, for the treatment of glaucoma.

Pharmaceutical Composition 3: Pharmaceutical Composition 1, for the prophylaxis of glaucoma.

Pharmaceutical Composition 4: Any One of Pharmaceutical Compositions 1 through 3, in a form adapted for direct administration to the vitreous tissue of the eye or for intravitreal or intraocular injection to the eye.

Pharmaceutical Composition 5: Pharmaceutical Composition 4, wherein the form is selected from the group consisting of an oil, a liquid and a suspension for direct application on the vitreous surface of the eye.

Pharmaceutical Composition 6: Any One of Pharmaceutical Compositions 1 through 3, formulated as a liquid or a suspension of the at least one vasodilator, wherein the vasodilator is a contact vasodilator.

Pharmaceutical Composition 7: Any One of Pharmaceutical Compositions 1 through 6, wherein the vasodilator is a calcium channel blocker.

Pharmaceutical Composition 8: Pharmaceutical Composition 7, wherein the at least one calcium channel blocker is a dihydropyridine selected from the group consisting of nifedipine, isradipine, felodipine, amlodipine, nicardipine, and clevidipine.

Pharmaceutical Composition 9: Pharmaceutical Composition 7, wherein the at least one calcium channel blocker is a non dihydropyridine selected from the group consisting of verapamil and diltiazem.

Pharmaceutical Composition 10: Any One of Pharmaceutical Compositions 1 through 6, wherein the vasodilator is an ACE inhibitor.

Pharmaceutical Composition 11: Pharmaceutical Composition 10, wherein the ACE inhibitor is selected from the group consisting of benazepril, captopril, enalapril, fosinopril, lisinopril, moexipril, perindopril, quinapril, ramipril, ramiprilat, and trandolapril.

Pharmaceutical Composition 12: Any One of Pharmaceutical Compositions 1 through 6, wherein the vasodilator is an angiotensin receptor blocker.

Pharmaceutical Composition 13: Pharmaceutical Composition 12, wherein the angiotensin receptor blocker is selected from the group consisting of azilsartan, candesartan, eprosartan, irbesartan, losartan, olmesartan, telmisartan, and valsartan.

Pharmaceutical Composition 14: Any One of Pharmaceutical Compositions 1 through 6, wherein the vasodilator is a nitrate.

Pharmaceutical Composition 15: Pharmaceutical Composition 14, wherein the nitrate is selected from the group consisting of nitroglycerin, isosorbide mononitrate and isosorbide dinitrate.

Pharmaceutical Composition 16: Any One of Pharmaceutical Compositions 1 through 6, wherein the vasodilator is an alpha blocker.

Pharmaceutical Composition 17: Pharmaceutical Composition 16, wherein the alpha blocker is selected from the group consisting of doxazosin, prazosin, and terazosin.

Pharmaceutical Composition 18: Any One of Pharmaceutical Compositions 1 through 6, wherein the vasodilator is a beta blocker.

Pharmaceutical Composition 19: Pharmaceutical Composition 18, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

Pharmaceutical Composition 20: Any One of Pharmaceutical Compositions 1 through 6, wherein the vasodilator is hydralazine.

Pharmaceutical Composition 21: Any One of Pharmaceutical Compositions 1 through 6, wherein the vasodilator is an angiotensin receptor-neprilysin inhibitor.

Pharmaceutical Composition 22: Pharmaceutical Composition 21, wherein the angiotensin receptor-neprilysin inhibitor is sacubitril/valsartan.

Pharmaceutical Composition 23: Any One of Pharmaceutical Compositions 1 through 22, wherein the concentration of the vasodilator is about 0.0001 mg per ml to 1000 mg per ml, optionally 1 mg per ml to 10 mg per ml, optionally 1 mg per ml to 1000 mg per ml, optionally 5 mg per ml to 10 mg per ml, optionally 10 mg per ml, optionally 20 mg per ml, optionally 30 mg per ml, optionally 60 mg per ml, optionally 90 mg per ml, optionally 120 mg per ml, optionally 180 mg per ml, optionally 240 mg per ml.

Pharmaceutical Composition 24: Any One of Pharmaceutical Compositions 1 through 2, wherein the concentration of the vasodilator is from about 0.0001% by weight to about 20% by weight, optionally about 0.01% by weight, optionally about 0.1% by weight, optionally about 1% by weight, optionally about 10% by weight, optionally about 20% by weight.

Method 25: A method for the treatment or prophylaxis of glaucoma in a patient in need thereof, comprising: administering an effective amount of the pharmaceutical composition according to any one of Pharmaceutical Compositions 1 through 24 to a patient in need thereof.

Method 26: Method 25, for the treatment of glaucoma.

Method 27: Method 25, for the prophylaxis of glaucoma.

Method 28: Method 25, wherein the composition is administered once a day, optionally two or more times a day, optionally once a week, optionally two or more times a week, optionally once a month, optionally two or more times a month, optionally a plurality of times a year.

Method 29: Method 25, wherein the composition is administered to a patient without symptoms of glaucoma related to vision loss. Such a patient may present with one or more of normal intraocular pressure, minor yet ophthalmoscopic-visible optic nerve damage, and cupping.

Method 30: Method 25, wherein the composition is administered to a patient that is at risk of developing glaucoma. If the patient tolerates oral drug compositions well, then the patient may be proscribed a long acting formulation. Side effects of some compositions provided in this disclosure may be relatively mild, even in oral formulations that are not directly applied to the eye. For example, side effects of calcium channel blockers may include Constipation, Dizziness, Fast heartbeat (palpitations), Fatigue, Flushing, Headache, Nausea, Rash, Swelling in the feet and lower legs.

### Example 13

In this example, a patient with a primary form of glaucoma is administered a topical formulation comprising ramiprilat to the eye. The administration of the topical formulation grows capillary networks and increases the capillary blood supply to the eye of the patient. As a result of the administration of the topical formulation, the patient is observed to have a reduction in intraocular pressure and repair to the optic nerve. The patient's primary form of glaucoma is therefore treated by the topical formulation.

### Example 14

In this example, a patient with a primary form of glaucoma is administered an injectable formulation comprising ramiprilat to the posterior chamber of the eye. The administration of the injectable formulation grows capillary networks and increases the capillary blood supply to the eye of the patient. As a result of the administration of the injectable formulation, the patient is observed to have a reduction in intraocular pressure and repair to the optic nerve. The patient's primary form of glaucoma is therefore treated by the topical formulation.

Any of the features the above referenced pharmaceutical compositions, uses, and methods is applicable to any other pharmaceutical composition, use, or method identified herein. Moreover, any of the features of the above referenced pharmaceutical compositions, uses, and methods is independently combinable, partly or wholly, with other embodiments of the pharmaceutical compositions, uses, and methods described herein in any way, e.g., one, two, or three or more features may be combinable in whole or in part. Further, any of the features of the pharmaceutical compositions, uses, and methods described above may be made optional to other pharmaceutical compositions, uses, and methods described herein. Any aspect or embodiment of a method or use described herein can be performed using a composition, e.g., a pharmaceutical composition and/or a compound as described herein, and any aspect or embodiment of a composition, e.g., a pharmaceutical composition and/or a compound described herein, can be used or adapted to perform a method or use as described herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more‘ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A pharmaceutical composition for the treatment or prophylaxis of glaucoma, comprising:
at least one vasodilator; and
at least one pharmaceutical excipient.

2. The pharmaceutical composition of claim 1, for the treatment of glaucoma.

3. The pharmaceutical composition of claim 1, for the prophylaxis of glaucoma.

4. The pharmaceutical composition of any one of claims 1 to 3, in a form for direct administration to the vitreous tissue of the eye or for intravitreal or intraocular injection to the eye.

5. The pharmaceutical composition of claim 4, wherein the form is selected from the group consisting of an oil, a liquid and a suspension for direct application on the vitreous surface of the eye.

6. The pharmaceutical composition of any one of claims 1 to 3, formulated as a liquid or a suspension of the at least one vasodilator, wherein the vasodilator is a contact vasodilator.

7. The pharmaceutical composition of any one of claims 1 to 6, wherein the vasodilator is a calcium channel blocker.

8. The pharmaceutical composition of claim 7, wherein the at least one calcium channel blocker is a dihydropyridine selected from the group consisting of nifedipine, isradipine, felodipine, amlodipine, nicardipine, and clevidipine.

9. The pharmaceutical composition of claim 7, wherein the at least one calcium channel blocker is a non dihydropyridine selected from the group consisting of verapamil and diltiazem.

10. The pharmaceutical composition of any one of claims 1 to 6, wherein the vasodilator is an ACE inhibitor.

11. The pharmaceutical composition of claim 10, wherein the ACE inhibitor is selected from the group consisting of benazepril, captopril, enalapril, fosinopril, lisinopril, moexipril, perindopril, quinapril, ramipril, ramiprilat, and trandolapril.

12. The pharmaceutical composition of claim 11, wherein the ACE inhibitor is ramipril or ramiprilat.

13. The pharmaceutical composition of claim 12, wherein the ACE inhibitor is ramiprilat.

14. The pharmaceutical composition of any one of claims 1 to 6, wherein the vasodilator is an angiotensin receptor blocker.

15. The pharmaceutical composition of claim 14, wherein the angiotensin receptor blocker is selected from the group consisting of azilsartan, candesartan, eprosartan, irbesartan, losartan, olmesartan, telmisartan, and valsartan.

16. The pharmaceutical composition of any one of claims 1 to 6, wherein the vasodilator is a nitrate.

17. The pharmaceutical composition of claim 16, wherein the nitrate is selected from the group consisting of nitroglycerin, isosorbide mononitrate and isosorbide dinitrate.

18. The pharmaceutical composition of any one of claims 1 to 6, wherein the vasodilator is an alpha blocker.

19. The pharmaceutical composition of claim 18, wherein the alpha blocker is selected from the group consisting of doxazosin, prazosin, and terazosin.

20. The pharmaceutical composition of any one of claims 1 to 6, wherein the vasodilator is a beta blocker.

21. The pharmaceutical composition of claim 20, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

22. The pharmaceutical composition of any one of claims 1 to 6, wherein the vasodilator is hydralazine.

23. The pharmaceutical composition of any one of claims 1 to 6, wherein the vasodilator is an angiotensin receptor-neprilysin inhibitor.

24. The pharmaceutical composition of claim 23, wherein the angiotensin receptor-neprilysin inhibitor is sacubitril/valsartan.

25. The pharmaceutical composition of any of claims 1 to 24, wherein the concentration of the vasodilator is about 0.0001 mg per ml to 1000 mg per ml, optionally 1 mg per ml to 10 mg per ml, optionally 1 mg per ml to 1000 mg per ml, optionally 5 mg per ml to 10 mg per ml, optionally 10 mg per ml, optionally 20 mg per ml, optionally 30 mg per ml, optionally 60 mg per ml, optionally 90 mg per ml, optionally 120 mg per ml, optionally 180 mg per ml, optionally 240 mg per ml.

26. The pharmaceutical composition of any of claims 1 to 24, wherein the concentration of the vasodilator is from about 0.0001% by weight to about 20% by weight, optionally about 0.01% by weight, optionally about 0.1% by weight, optionally about 1% by weight, optionally about 10% by weight, optionally about 20% by weight.

27. A method for the treatment, prevention, reduction, or prophylaxis of glaucoma in a patient in need thereof, comprising:
administering an effective amount of the pharmaceutical composition according to any one of claims 1 to 26 to a patient in need thereof.

28. The method of claim 27, for the treatment of glaucoma.

29. The method of claim 27, for the prophylaxis of glaucoma.

30. The method of any one of claims 27 to 29, wherein the composition is administered once a day, optionally two or more times a day, optionally once a week, optionally two or more times a week, optionally once a month, optionally two or more times a month, optionally a plurality of times a year.

31. The method of any one of claims 27 to 30, wherein composition is topically administered to the eye of the patient in need.

32. The method of any one of claims 27 to 31, wherein topically administered is by ophthalmic drops.

33. The method of any one of claims 27 to 32, wherein administering an effective amount of the pharmaceutical composition according to claim 1 provides capillary formation in the patient in need thereof.

34. The method of any one of claims 27 to 33, wherein the administering an effective amount of the pharmaceutical composition according to claim 1 to a patient in need thereof, reduces the interocular pressure of the eye.

35. A method for the reducing one or more symptoms of glaucoma in a patient in need thereof, comprising:
administering an effective amount of the pharmaceutical composition according to any one of claims 1 to 26 to a patient in need thereof.

36. The method of claim 35, wherein the composition is administered once a day, optionally two or more times a day, optionally once a week, optionally two or more times a week, optionally once a month, optionally two or more times a month, optionally a plurality of times a year.

37. The method of claim 35 or 36, wherein composition is topically administered to the eye of the patient in need.

38. The method of any one of claims 35 to 37, wherein topically administered is by ophthalmic drops.

39. The method of any one of claims 35 to 38, wherein administering an effective amount of the pharmaceutical composition according to claim 1 provides capillary formation in the patient in need thereof.

40. The method of any one of claims 35 to 39, wherein the administering an effective amount of the pharmaceutical composition according to claim 1 to a patient in need thereof, reduces the interocular pressure of the eye.

41. A method of growing a capillary network and increasing the capillary blood supply in the eye of a subject, the method comprising:
administering a pharmaceutical composition comprising ramipril or ramiprilat and a pharmaceutically acceptable excipient to the eye of the subject,
thereby treating multiple forms of glaucoma.

42. The method of claim 41, wherein the pharmaceutical composition comprises ramiprilat.

43. The method of claim 41 or 42, wherein administering the pharmaceutical composition repairs the optic nerve of the subject.

44. The method of any one of claims 41 to 43, wherein the multiple forms of glaucoma is a primary glaucoma.

45. The method of claim 44, wherein the primary glaucoma is open-angle glaucoma, normal-tension glaucoma, angle-closure glaucoma, congenital glaucoma.

46. The method of any one of claims 41 to 43, wherein the multiple forms of glaucoma is a secondary glaucoma.

47. The method of claim 46, wherein the secondary glaucoma is neovascular glaucoma, pigmentary glaucoma, exfoliation glaucoma, or uveitic glaucoma.

48. The method of any one of claims 41 to 47, wherein the pharmaceutical composition is administered topically to the eye of the subject.

49. The method of claim 48, wherein the pharmaceutical composition is in the form of a gel, cream, or eye drops.

50. The method of any one of claims 41 to 47, wherein the pharmaceutical composition is administered by intravitreal injection or direct application to the tissue of the eye.
